(19) United States
(12) Patent Application Publication
(10) Pub. No.: US 2023/0323129 A1
(43) Pub. Date: Oct. 12, 2023

(54) USE OF AN ACTIVATED CARROT FIBER FOR PRODUCING PRODUCTS

(71) Applicant: Herbstreith & Fox GmbH & Co. KG Pektin- Fabriken, Neuenbürg (DE)

(72) Inventor: Gerhard F. Fox, Neuenbürg (DE)

(21) Appl. No.: 18/006,165

(22) PCT Filed: Jul. 22, 2021
(86) PCT No.: PCT/EP2021/070480

§ 371 Date: Jan. 20, 2023

(30) Foreign Application Priority Data
Jul. 22, 2020 (DE) 10 2020 119 364.5
Oct. 2, 2020 (DE) 10 2020 125 835.6

(51) Int. Cl. C08L 99/00; A23L 33/24; A23L 29/262; A23K 10/37; A23K 20/163; B27N 3/04; D01C 1/00; B01J 20/24; C11D 3/382; C09D 5/00; C09D 7/65; C09D 7/40; C06B 23/00; C10M 169/02; C10M 121/00; C08J 5/04; C04B 18/24; C09J 11/08; C05G 5/40; C05G 5/14; C09K 8/035; C09K 8/70; A61Q 19/00; A61K 47/46; A61L 15/40; A01K 1/015

(52) U.S. Cl. CPC C08L 99/00; C10N 2050/10; A23L 29/262; A23K 10/37; A23K 20/163; B27N 3/04; D01C 1/00; B01J 20/24; C11D 3/382; C09D 5/00; C09D 7/65; C09D 7/70; C06B 23/001; C10M 169/02; C10M 121/00; C08J 5/045; C04B 18/248; C09J 11/08; C05G 5/40; C05G 5/14; C09K 8/035; C09K 8/70; A61Q 19/00; A61K 47/46; A61L 15/40; A01K 1/0155; D10B 2401/06; D10B 2201/01; A23L 33/24

(57) ABSTRACT

The present invention relates to the use of an activated carrot fiber for preparing products in the food or non-food area. The invention also relates to products containing the activated carrot fiber.

The present invention relates to the use of an activated carrot fiber for the manufacturing of products in the food or non-food area. The invention also relates to products containing the activated carrot fiber.

### BACKGROUND OF THE INVENTION

Dietary fibers are largely non-digestible food components, mostly carbohydrates, which predominantly exist in plant-based foods. For purposes of simplicity, dietary fibers are divided into water-soluble dietary fibers, such as pectin, and water-insoluble dietary fibers, such as e. g. cellulose. Dietary fibers are considered to be an important part of the human diet.

Thus, the consumption of dietary fibers is considered to promote health. The use of fruit fibers, such as sugar beet, apple or citrus fibers, as dietary fibers in the production of food is gaining increasing importance. One reason is that fruit fibers are mixtures of insoluble dietary fibers such as cellulose and soluble dietary fibers such as pectin, resulting in an ideal spectrum of health-promoting activity. The functional properties of food products may be modified by use of fruit fibers like citrus fibers or apple fibers. Nowadays, fruit fibers are also employed in non-food products.

Thus, U.S. Pat. No. 5,964,983 teaches the use of a microfibrillar cellulose produced from sugar beets as a thickener for colors or drilling fluids. The method disclosed in U.S. Pat. No. 5,964,983, however, requires great effort since it comprises an acid or alkaline extraction, followed by an aqueous washing step, pressure homogenization, an ethanolic washing step and drying. In addition, the fiber properties are shown to change substantially, depending on the production method, consequently also determining usability for the optimization of food or non-food products.

Thus, there is a demand for improved pectin-containing fruit fibers and the new or improved possibilities of their usage resulting therefrom.

The objective of the present invention thus is to provide an improvement or an alternative to the prior art.

### SUMMARY OF THE INVENTION

In a first aspect of the present invention, the objective is achieved by use of an activated carrot fiber for manufacturing a product, selected from the group consisting of food products, feedstuff, commodity goods, animal need, hygiene products, personal care products, cleaning agents, coating agents, care agents, explosives, lubricants, cooling agents, plastic products, fabrics, imitation leather, varnish, ink, paints, building materials, composite materials, paper, cardboard, adhesive, fertilizers, drugs, medical products, batteries, wherein the activated carrot fiber has a viscosity of 800 mPas to 5000 mPas at a shear rate of τ=50 1/s in a 2.5 wt % aqueous suspension.

The production method described in the following results in activated carrot fibers with a large internal surface, which also increases water-binding capacity and contributes to a good viscosity formation.

These fibers are activated fibers with sufficient firmness in an aqueous suspension, so that no additional shear forces are required in application for the user to obtain optimal rheological properties like viscosity or texturing.

The inventors have found that the activated carrot fibers produced with the method described below to exhibit good rheological characteristics. The fibers according to the invention can be easily rehydrated, and the advantageous rheological properties remain even after rehydration.

The production method described in the following results in activated carrot fibers which are to a large degree without taste and smell, which makes them advantageously usable for the application in the food industry. The individual taste of the other ingredients is not masked and can therefore develop in an optimum manner.

Additionally, the activated carrot fiber used according to the invention has a more potent effectiveness. Compared to modified starch, less than half the amount needs to be employed to produce a fat-containing cream with comparable baking stability.

The activated carrot fiber used according to the invention is a non-digestible dietary fiber low in calories. Dietary fibers are an important part of the human diet.

The activated carrot fibers used according to the invention are obtained from carrots, thus representing natural ingredients with well-known positive characteristics.

Plant-based processing residues, such as carrot pulp, can be used as the raw material in the production method described below. Such processing residues are inexpensive, available in sufficient amounts and form an ecologically sustainable source of the activated carrot fibers according to the invention.

Carrot fibers are well-established and accepted in the food industry so that respective compositions can be immediately used without lengthy admission procedures, even internationally.

### DETAILED DESCRIPTION OF THE INVENTION

In a second aspect, the invention relates to the use of an activated carrot fiber in the field of construction, in extraction by drilling of boreholes and in the agricultural field, the activated carrot fiber having a viscosity of 800 mPas to 5000 mPas at a shear rate of τ=50 1/s in a 2.5 wt % aqueous suspension.

In the uses taught above, the activated carrot fiber according to the invention can have one or more of the following functions: as a foaming agent, a whipping agent, a release agent, a free flow agent, a stabilizer, an emulsifier, a carrier, a filler, a texturing agent, a thickener, a gelling agent, a solidifying agent, a dietary fiber, a reinforcing agent, a humectant, a filter aid, an egg substitute, a glazing agent, an improving agent for freeze-thaw stability and an improving agent for baking stability.

The invention relates to the use of an activated carrot fiber. Such an activated carrot fiber can be obtained from carrot pulp that is disintegrated as starting material by incubation of an aqueous carrot pulp suspension.

### THE ACTIVATED CARROT FIBER

According to the invention, an activated carrot fiber is employed.

The activated carrot fiber has a yield point II (rotation) of between 15 and 30 Pa, advantageously between 17.5 and 27.5 Pa and particularly advantageously between 20 and 25 Pa in a 2.5 wt % aqueous suspension. Advantageously, this activated carrot fiber is obtainable or obtained by the production method described below.

The activated carrot fiber has a yield point I (rotation) of between 15 and 30 Pa, advantageously between 17.5 and 27.5 Pa and particularly advantageously between 20 and 25 Pa in a 2.5 wt % aqueous suspension. Advantageously, this activated carrot fiber is obtainable or obtained by the production method described below.

The activated carrot fiber has a yield point II (cross-over) of between 20 and 35 Pa, advantageously between 22.5 and 32.5 Pa and particularly advantageously between 25 and 30 Pa in a 2.5 wt % aqueous suspension. Advantageously, this activated carrot fiber is obtainable or obtained by the production method described below.

The activated carrot fiber has a yield point I (cross-over) of between 25 and 35 Pa, advantageously between 20 and 30 Pa and particularly advantageously between 22.5 and 27.5 Pa in a 2.5 wt % aqueous suspension. Advantageously, this activated carrot fiber is obtainable or obtained by the production method described below.

In a 2.5 wt % aqueous suspension, the activated carrot fiber has a dynamic Weissenberg number of between 5 and 11 Pa, advantageously between 6 and 10 Pa and particularly advantageously between 7 and 9 Pa. Advantageously, this activated carrot fiber is obtainable or obtained by the production method described below.

In a 2.5 wt % aqueous suspension, the activated carrot fiber has a dynamic Weissenberg number of between 5 and 11 Pa, advantageously between 6 and 10 Pa and particularly advantageously between 7 and 9 Pa. Advantageously, this activated carrot fiber is obtainable or obtained by the production method described below.

In an advantageous embodiment, the activated carrot fiber has, in a 4 wt % aqueous suspension, a firmness of between 320 g and 510 g, advantageously between 350 g and 480 g and particularly advantageously between 380 and 450 g. Preferably, the activated carrot fiber characterized by this firmness is obtainable or obtained by the production method described below.

Preferably, the activated carrot fiber has a viscosity of from 800 to 4800 mPas, preferably 1000 to 4500 mPas and particularly preferably 1200 to 4000 mPas, wherein the activated carrot fiber is dispersed in water as a 2.5 wt % solution and the viscosity is measured with a shear rate of 50 5-1 at 20° C. For example, the activated carrot fiber can have a viscosity of 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300 or 4400 mPas. Preferably, the activated carrot fiber characterized by this viscosity is obtainable or obtained by the production method described below.

For determining viscosity, the carrot fiber is dispersed in demineralized water with the method disclosed in the examples as a 2.5 wt % solution, and viscosity is determined at 20° C. and four shearing sections (first and third section =constant profile; second and fourth section =linear ramp; each measurement at a shear rate of 50 s⁻¹) (rheometer; Physica MCR series, measuring bob CC25 [corresponding to Z3 DIN], Anton Paar company, Graz, Austria). The advantage of an activated carrot fiber with such a high viscosity is that a lower amount of fiber is necessary for thickening the final product. In addition, the fiber thus creates a creamy texture.

The activated carrot fiber advantageously has a water binding capacity of between 25 and 50 g/g, preferably between 30 and 45 g/g, particularly preferably between 32.5 and 42.5 g/g and especially preferably between 35 and 40 g/g. Such an advantageously high water-binding capacity leads to a high viscosity and consequently also to a lower fiber consumption with a creamy texture. Preferably, the activated carrot fiber characterized by this water binding capacity is obtainable or obtained by the production method described below.

According to one embodiment, the activated carrot fiber has a moisture of less than 15%, preferably less than 10% and particularly preferably less than 8%. Preferably, the activated carrot fiber characterized by this moisture is obtainable or obtained by the production method described below.

It is also preferable for the activated carrot fiber to have, in a 1.0% aqueous solution, a pH value of 3.5 to 5.0 and preferably 3.9 to 4.5. Preferably, the activated carrot fiber characterized by this range of pH values is obtainable or obtained by the production method described below.

The activated carrot fiber advantageously has a particle size in which at least 90% of the particles are smaller than 400 μm, preferably smaller than 350 μm and in particular smaller than 300 μm. Preferably, the activated carrot fiber characterized by this particle size is obtainable or obtained by the production method described below.

In one advantageous embodiment, the activated carrot fiber has a lightness value of L*>90, preferably L*>91 and particularly preferably L*>92. Thus, the activated carrot fibers are nearly colorless and do not lead to any significant coloring of the food products they are used in. Preferably, the activated carrot fiber characterized by this lightness value is obtainable or obtained by the production method described below.

Advantageously, the activated carrot fiber has a dietary fiber content of 80 to 95%. Preferably, the activated carrot fiber characterized by this dietary fiber content is obtainable or obtained by the production method described below.

The activated carrot fiber used according to the invention is preferably present in powder form. The advantage is that in this manner, there is a formulation with low weight and long shelf life which is also easy to employ in process technology. This formulation is only made possible by the activated carrot fiber used according to the invention which, other than modified starches, does not tend to lump formation when it is dissolved in liquids.

### PRODUCTION OF THE ACTIVATED CARROT FIBER

The activated carrot fiber is obtainable by a method comprising the following steps:
(a) providing a carrot-containing plant mass in the form of dry mass or wet mass;
(b) in case of dry mass, hydration by incubation of the dry pulp with an aqueous liquid and separation of the aqueous liquid from the hydrated dry mass;
(c) optional enzymatic treatment of the wet mass from step (a) or of the hydrated dry mass from step (b) in aqueous suspension with cellulase and/or with pectin methyl esterase to obtain an enzymatically treated material;
(d) washing the wet mass from step (a), the hydrated dry mass from step (b) or the enzymatically treated material from step (c) at least twice with an organic solvent and subsequently separating the washed material from the organic solvent;
(e) drying the washed material from step (d), comprising drying at normal pressure or vacuum drying to obtain the activated carrot fiber.

The inventors have surprisingly found that the method according to the invention results in activated carrot fibers without the necessity of performing the activation methods otherwise necessary, such as the application of shear forces or disintegration in an acidic environment at increased temperatures.

As raw material, a carrot-containing plant mass, and preferably processing residues of carrots, can be employed.

One possibility of using this carrot-containing plant mass is in the form of dry mass, for example as carrot dry pulp. In the context of the invention, a dry mass is understood to be a carrot-containing plant mass which has less than 15%, preferably less than 10% and further preferably less than 8% moisture. The use of dry plant material allows a production all year round.

In case the carrot-containing plant mass is present in the form of a dry mass, it must be hydrated by incubation with an aqueous liquid according to step (b). During this process, the plant mass forms a slurry of the carrot pieces or carrot particles in the aqueous solution. This slurry is a suspension since there is a heterogeneous mixture of a liquid and carrot particles (preferably finely) distributed therein. Since the suspension tends to sedimentation and phase separation, the particles are suitably kept in suspension by shaking or stirring. That is, there is no dispersion in which the particles are comminuted by mechanical action (shearing) so as to be finely dispersed. After incubation with the aqueous liquid, the hydrated dry mass is separated from the aqueous solution by solid-liquid separation. This is preferably done by means of a decanter. Alternative separation methods are a sieve drum, a separator, a sedicanter or a press.

In step (c), the carrot-containing plant mass can be subjected to enzymatic treatment. This treatment comprises de-esterification of the high methoxyl pectin present in the carrot material by means of a pectin methyl esterase and/or partial degradation of the cellulose present in the carrot material by means of a cellulase.

In a further step (d), the enzymatically treated material or the hydrated dry material from step (b) or the carrot-containing plant material provided as wet mass from step (a) is washed several times, i.e. at least twice, with an organic solvent. This multi-stage washing with alcohol first causes an improvement of the functional fiber properties and thus contributes decisively to the activation of the carrot fiber. In addition, undesired accompanying substances are removed from the material, thus ensuring sensory neutrality of the end product. This applies to both olfactory and gustatory substances.

Drying from the alcoholic phase in step (e) is essential for the future functional properties, since the fibers dry with open pores, resulting in good swelling and wetting properties that would not be present if they were dried from the aqueous phase, since the individual fibers would then crust over via hydrogen bonds. The dried fiber material produced according to the process described herein is an activated carrot fiber, insofar an open-pore fiber with good swelling and wetting properties results, which is also expressed in advantageous functional properties such as viscosity, water-binding capacity and firmness.

As raw material or starting material, a carrot-containing plant mass and preferably processing residues of carrots are used.

In this regard, the skilled person can make use of a wide variety of carrot materials. In one embodiment, the carrot-containing plant mass is selected from the group consisting of carrot pulp, carrot flour, carrot pulp flour, carrot semolina and carrot puree, although a mixture of the aforementioned masses may also be used.

A “carrot-containing vegetable mass” according to the invention is intended to mean shredded carrots, so that no whole carrots are used, but at least carrot pieces, carrot semolina, or even finely particulate carrots in the form of carrot flour.

According to the invention, “carrot pulp” is defined to be the shredded solid residues produced during carrot processing. The processing typically involves juice production. Here, the carrot pulp initially accumulates as wet pulp. To obtain a pulp material with improved storability, the pulp is usually dried and can then be stored and further used as dry pulp.

During hydration in step b), the dry matter is rehydrated by contacting and incubating it with an aqueous liquid, thus preparing it for the following processing steps. The mixture of carrot dry matter to be hydrated and aqueous liquid is also referred to as an aqueous incubation solution in the following.

During hydration in step b), incubation with the aqueous solution takes place at a temperature between 20° C. and 70° C., advantageously at a temperature between 25° C. and 65° C., and particularly advantageously at a temperature between 30° C. and 60° C. Especially an increased temperature accelerates the rehydration.

The aqueous liquid used in the hydration in step b) can be an aqueous buffer or water. To set controlled hydration conditions, the use of demineralized water is preferred.

Expediently, hydration in step b) is carried out by incubation with the aqueous liquid for a period of time ranging from 10 min to 4 hours, advantageously for a period of time ranging from 20 min to 3 hours, and particularly advantageously for a period of time ranging from 30 min to 2 hours.

During hydration in step b), the dry matter in the aqueous incubation solution is between 0.25 wt % and 20 wt %, preferably between 0.5 wt % and 15 wt %, and particularly preferably between 1 wt % and 10 wt %.

The hydration in step b) is advantageously carried out under stirring or shaking of the aqueous suspension. This accelerates the hydration process and contributes to a more uniform hydration.

Following incubation with the aqueous liquid, the dry mass hydrated in step b) is separated from the aqueous solution by solid-liquid separation. This is preferably done by a decanter. Alternative separation methods include a sieve drum, a separator, a sedicanter or a press.

According to one embodiment, the carrot-containing plant material may be subjected to an enzymatic treatment with a pectin methyl esterase in step (c) of the method, which is also synonymously referred to as “enzymatic de-esterification”.

The material contained in the plant fiber is typically high methoxyl pectin.

A pectin according to the application is defined to be a plant polysaccharide which, as a polyuronide, consists essentially of α-1,4-glycosidically linked D-galacturonic acid units. The galacturonic acid units are partially esterified with methanol. The degree of esterification describes the percentage of carboxyl groups in the galacturonic acid units of pectin which are present in esterified form, e.g. as methyl esters.

According to the invention, a high methoxyl pectin is understood to be a pectin that has a degree of esterification of at least 50%, whereas a low methoxyl pectin has a degree of esterification of less than 50%. The degree of esterification describes the percentage of carboxyl groups in the galacturonic acid units of the pectin which are present in esterified form, e.g. as methyl esters. The degree of esterification can be determined using the method according to JECFA (Monograph 19-2016, Joint FAO/WHO Expert Committee on Food Additives).

Pectin methyl esterase hydrolyzes the methyl esters of the galacturonic acid groups in pectin thereby forming poly-galacturonic acid and methanol. The resulting low methoxyl pectins can form a gel in the presence of polyvalent cations, even in the absence of sugar, and can also be used in a wide pH range.

A pectin methyl esterase (abbreviation: PME, EC 3.1.1.11, also called pectin demethoxylase, pectin methoxylase) is a commonly found enzyme in the cell walls of all higher plants and some bacteria and fungi that cleaves the methyl esters of pectins, thereby forming poly-galacturonic acid and releasing methanol. PME has been isolated in many isoforms, all of which can be used for enzymatic de-esterification according to the invention. Thus, PME has been isolated in many isoforms from plant pathogenic fungi such as Aspergillus foetidus and Phytophthora infestans as well as from higher plants, e.g. tomatoes, potatoes and oranges. Fungal PMEs exhibit optimal activity between pH 2.5 and 5.5, whereas plant PMEs exhibit pH optima between pH 5 and 8. The relative molecular mass is between 33,000 and 45,000. The enzyme is present as a monomer and is glycosylated. The Km value ranges from 11 to 40 mM pectin for fungal PME and from 4 to 22 mM pectin for plant PME. The commercially available preparations of PME are obtained either from the supernatants of fungal mycelial cultures or, in the case of plants, from fruits (peels of oranges and lemons, tomatoes). The preferred pectin methyl esterases have a pH optimum between 2 and 5 and a temperature optimum at 30 to 50° C., although depending on the enzyme, appreciable enzyme activity can be observed above a temperature as low as 15° C.

The following table gives some examples of commercially available PMEs with their reaction optima:

| Product name | Manufacturer | Optima |
|---|---|---|
| Rapidase PEP | DSM | pH = 4-5; T = 50° C. |
| Pectinase 872 L | Biocatalysts | pH = 4-5; T = 30-50° C. |
| Pectinesterase VP 1508/14 | Erbsloh | pH = 4-5; T = 30-50° C. |

For enzymatic de-esterification, at least one pectin methyl esterase (EC 3.1.1.11) is added to the aqueous suspension. In one embodiment, exactly one isoform of a PME is added to the suspension. Alternatively, a mixture of different isoforms may be used.

Preferably, the pectin methyl esterase is added to the aqueous suspension in such a way that a total PME activity of 1000 to 10,000 units/l, advantageously of 3000 to 7500 units/l, and particularly advantageously of 4000 to 6000 units/1 results. For example, the total PME activity may be 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200 or 7400 Units/l.

In the enzymatic de-esterification in step c), the incubation with the at least one pectin methyl esterase in the aqueous suspension is carried out for a period of time ranging from one to 10 hours and preferably from 2 to 5 hours.

The person skilled in the art will adjust the temperature to the PME isoform used. Typically, the enzymatic treatment in step c) is carried out at a temperature of between 10° C. and 70° C., preferably between 20° C. and 60° C., and particularly preferably between 30° C. and 50° C.

Accordingly, the person skilled in the art will also adjust the optimum pH value for the de-esterification depending on the particular pectin methyl esterase used. Preferably, a pH value of between 3.5 and 5.5 and particularly preferably between 4.0 and 5.0 is provided here. If necessary, the pH is adjusted for this purpose by adding an acid or a buffer system operating in an acidic environment prior to enzymatic de-esterification.

To achieve an acidic pH, the person skilled in the art can make use of any acid or acidic buffer solution known to him. For example, an organic acid such as citric acid can be used. Alternatively, or in combination, a mineral acid can also be used. Examples include sulfuric acid, hydrochloric acid, nitric acid or sulfurous acid. Preferably, sulfuric acid is used.

For satisfactory de-esterification in step c), the dry matter content in the aqueous suspension must not be too high and should advantageously be below 10 wt %. In one embodiment, the dry matter content is between 0.5 wt % and 6 wt %, preferably between 1 wt % and 4 wt %, and particularly preferably between 2 wt % and 3wt %.

The enzymatic de-esterification in step c) can be carried out with stirring or shaking of the aqueous suspension, taking care that the enzyme does not foam up. This is preferably done in a continuous manner so that the particles of the suspension are kept in suspension.

The carrot-containing mass is present as an aqueous suspension during the enzymatic de-esterification in step c). According to the invention, a suspension is a heterogeneous mixture of substances consisting of a liquid and solids (carrot particles) finely distributed therein. Since the suspension tends to sedimentation and phase separation, the particles are suitably kept in suspension by shaking or stirring. Thus, there is no dispersion in which the particles are comminuted by mechanical action (shear) so that they are finely dispersed.

According to one embodiment, the carrot-containing plant material may be subjected to an enzymatic treatment with a cellulase in step (c) of the method, which is also synonymously referred to as “enzymatic cellulose hydrolysis”.

A cellulase is an enzyme capable of cleaving the β-1,4-glycosidic bond of cellulose through which glucose is released. Carrot material is largely composed of cellulose, which is fragmented accordingly by cellulase treatment. Cellulase treatment has been found to surprisingly improve carrot fiber functionality in terms of water binding and viscosity build-up.

The group of cellulases consists of three different enzyme types whose interaction enables rational digestion of the long-chain cellulose molecules (3000-15000 chained glucose molecules): endoglucanases (EC 3.2.1.4) cleave cellulose in larger portions. Endoglucanases as the first type of enzymes are the only ones able to work within cellulose chains, but only within so-called amorphous regions, where the cellulose molecules are disordered to each other and thus do not build up crystalline regions. As a result, they generate a larger number of chain ends. Many molecules of the second enzyme type, the exoglucanases (EC 3.2.1.91), can then work on these simultaneously and continuously shorten the cellulose chains by always cleaving two sugar molecules as a double sugar (disaccharide) cellobiose. The molecules of the third type of enzyme, cellobiase or β-glucosidase (EC 3.2.1.21), can thus again work simultaneously and, as a conclusion of the decomposition process, finally hydrolyze the β-glycosidic link between the two glucose molecules of cellobiose, thus releasing two glucose molecules.

Cellulase is added to the aqueous suspension for enzymatic cellulose hydrolysis. In one embodiment, exactly one type of cellulase enzyme may be added, i.e., either an endoglucanase (EC 3.2.1.4), an exoglucanase (EC 3.2.1.91), or a β-glucosidase (EC 3.2.1.21).

In a preferred embodiment, two or further preferably all three of the aforementioned cellulase enzyme types are used.

The cellulase treatment in step c) expediently leads to only partial hydrolysis of the cellulose present in the carrot mass. Excessive hydrolysis leads to irreversible degradation of the cellulose in the fiber material, which has a detrimental effect on fiber functionality.

Expediently, during the enzymatic cellulose hydrolysis in step c), the aqueous suspension contains the cellulase, or the cellulase mixture, in a total activity of from 100 to 3000 units/l, advantageously from 150 to 2000 units/l, further advantageously from 200 to 1000 units/l, and particularly advantageously from 250 to 400 units/l. For example, the total cellulase activity may be 100, 150, 200, 250, 300, 350, 400, 450, 500, 550, 600, 650, 700, 750, 800, 850, 900, 950, 1000, 1200, 1400, 1600, 1800, 2000, 2200, 2400, 2600 or 2800 Units/l.

In one embodiment, the incubation with cellulase in the aqueous suspension is carried out for a period of time ranging from 30 min to 4 hours and preferably from one to 3 hours.

The person skilled in the art will adjust the temperature to the cellulase used in step c). Typically, the cellulose hydrolysis takes place at a temperature of between 30° C. and 80° C., preferably between 35° C. and 75° C., and particularly preferably between 40° C. and 70° C.

Accordingly, the person skilled in the art will also set the optimum pH value for cellulose hydrolysis in step c) depending on the cellulase used in each case. Preferably, a pH value of between 3.0 and 7.0 is provided here, and particularly preferably between 3.5 and 6.0. If necessary, the pH is adjusted for this purpose before enzymatic cellulose hydrolysis by adding an acid or a buffer system operating in an acidic environment.

To achieve an acidic pH, the person skilled in the art can make use of any acid or acidic buffer solution known to him. For example, an organic acid such as citric acid can be used. Alternatively, or in combination, a mineral acid can also be used. Examples include sulfuric acid, hydrochloric acid, nitric acid or sulfurous acid. Preferably, sulfuric acid is used.

For satisfactory hydrolysis of the cellulose in step c), the content of dry matter in the aqueous suspension must not be too high and should advantageously be below 10wt %. In one embodiment, the dry matter content is between 0.5 wt % and 6 wt %, preferably between 1 wt % and 4 wt %, and particularly preferably between 2 wt % and 3wt %.

The enzymatic cellulose hydrolysis in step c) can be carried out with stirring or shaking of the aqueous suspension, taking care that the enzyme does not foam up. This is preferably done in a continuous manner so that the particles of the suspension are kept in suspension

The carrot-containing mass is present as an aqueous suspension during the enzymatic hydrolysis of the cellulose in step c). According to the invention, a suspension is a heterogeneous mixture of substances consisting of a liquid and solids (raw material particles) finely distributed therein. Since the suspension tends to sedimentation and phase separation, the particles are suitably kept in suspension by shaking or stirring. Thus, there is no dispersion in which the particles are comminuted by mechanical action (shear) so that they are finely dispersed.

According to one embodiment, the carrot-containing plant material may be enzymatically treated with either pectin methyl esterase or, alternatively, cellulase.

In an advantageous embodiment, the carrot-containing plant material is enzymatically treated with both pectin methyl esterase and cellulase. Here, the enzymatic treatment with cellulase and pectin methyl esterase can be carried out simultaneously or sequentially.

In step (d), a washing step is then carried out using an organic solvent, preferably a water-miscible organic solvent. This involves washing at least twice with the organic solvent.

The organic solvent is advantageously an alcohol, which may preferably be selected from the group consisting of methanol, ethanol and isopropanol.

The washing step d) is advantageously carried out at a temperature of between 40° C. and 75° C., preferably between 50° C. and 70° C., and particularly preferably between 60° C. and 65° C.

Contacting with the organic solvent advantageously takes place between 60 min to 10 hours, preferably between 2 hours to 8 hours.

Each step of washing with the organic solvent in step (d) comprises contacting the material with the organic solvent for a specific duration of time, followed by separation of the material from the organic solvent. For this separation, preferably a decanter or a press is used.

During washing with the organic solvent in step (d), the dry mass in the washing solution advantageously amounts to between 0.5 wt % and 15 wt %, preferably between 1.0 wt % and 10 wt % and particularly preferably between 1.5 wt % and 5.0 wt %.

Washing with organic solvent according to step (d) is preferably performed with mechanical movement of the washing mixture. This is preferably done in a tank with a stirring unit.

For washing with the organic solvent in step (d), advantageously a device for homogenization of the suspension is used. This device is preferably a toothed ring disperser.

According to an advantageous embodiment, washing with the organic solvent in step (d) takes place in a counterflow procedure.

In one embodiment, partial neutralization by addition of Na or K salts, NaOH or KOH, takes place during washing in step (d) with the organic solvent.

During washing with the organic solvent in step d), decoloring of the material can be performed in addition. This can be done by adding one or more oxidants. Examples of oxidants are chlorine dioxide and hydrogen peroxide which can be used by themselves or in combination.

In an advantageous embodiment, during the at least two-fold washing with an organic solvent in step d), the concentration of the organic solvent in the solution increases with each washing step. By this incremental increase in organic solvent, the portion of water in the fiber material is reduced in a controlled manner such that the rheological properties of the fibers are maintained during the subsequent steps of solvent removal and drying, and the activated fiber structure does not collapse.

Preferably, the final concentration of the organic solvent amounts to between 60 and 70 vol % in the first washing step, between 70 and 85 vol % in the second washing step, and in an optional third washing step, between 80 and 90 vol %.

In step (e), the washed material from step (d) is dried, wherein the drying process in one embodiment comprises and preferably consists of vacuum drying. During vacuum drying, the washed material as drying material is subjected to a vacuum, which lowers the boiling point, thus leading to evaporation of the water even at low temperatures. The evaporation heat, which is continuously lost by the drying material, is suitably restored from outside up to constant temperature. The effect of vacuum drying is that it lowers the equilibrium vapor pressure, promoting capillary transport. This has in particular been proven advantageous for the present carrot fiber material since in this manner the activated open fiber structures and the resulting rheological properties are maintained. Preferably, vacuum drying takes place at a negative pressure of less than 400 mbar, preferably less than 300 mbar, further preferably less than 250 mbar and particularly preferably less than 200 mbar.

Drying under vacuum in step (e) advantageously takes place at a shell temperature of between 40° C. and 100° C., preferably between 50° C. and 90° C. and particularly preferably between 60° C. and 80° C. After drying, the product is expediently cooled to ambient temperature.

In an alternative embodiment, the washed material from step (d) is dried in step (e), wherein drying comprises drying under normal pressure. Examples of suitable drying methods are fluid-bed drying, fluidized-bed drying, belt drying, rotary drum drying or paddle drying. Fluidized-bed drying is particularly preferable. The advantage of this method is that the product is dried while being in a loose state which facilitates a subsequent optional milling step. In addition, this type of drying avoids damage to the product by local overheating since the input of heat can be dosed very well.

Drying under normal pressure in step (e) can take place at a temperature of between 50° C. and 130° C., preferably between 60° C. and 120° C. and particularly preferably between 70° C. and 110° C. After drying, the product is expediently cooled to ambient temperature.

In an advantageous embodiment, the method additionally comprises a comminuting, milling or sieving step after drying in step (e). This step is advantageously performed such that as a result, 90% of the particles have a size of less than 400 μm, preferably less than 350 μm and particularly preferably less than 300 μm. At this particle size, the fiber is well dispersible and has optimum swelling properties.

The activated carrot fiber used according to the invention, as well as a method for the production thereof, are disclosed in the application DE 10 2020 119 364.5.

In one embodiment, the activated carrot fiber can be used for manufacturing of a food product. This can be any food product known to the person skilled in the art. Advantageously, the food product is selected from the group consisting of preserved products, deep-frozen foods, vegan food, vegetarian food, gluten-free food, low-calorie food, low-sugar food, lactose-free food, jelly, jelly-type sweets, sauce, granola bars, fruit pieces, fruit snacks, fruit bars, milk substitute drink, milk substitute product, foam sugar products, sherbet, ice cream, desserts, fermented drink, milk product, delicacies, fruit drink, fruit drink containing alcohol, cocktail, vegetable drink, chutney, barbecue sauce, smoothies, instant drink, fruit spread, fruit compote, fruit dessert, fruit sauce, fruit preparations, bake-stable fruit preparations, fruit preparations for yoghurt, bake-stable vegetable preparations, bake-stable fatty fillings, baked goods, pasta and pasta fillings, noodle dishes, potato snack, cheese and cream cheese preparations, meat substitute products, extruder products, corn flakes, breakfast cereals, soup, sauce, mayonnaise, meats, sausages, sausage casings, seafood, spirits, lozenges, functional food, nutritional supplements and dietary foods such as enteral foods, dysphagia food or sip feed.

In the food area, the activated carrot fiber is especially suited for textured products. For further optimization, it can be combined here with hydrocolloids and/or functional dietary fibers.

In milk substitution drinks, such as, for example, almond milk, it has been shown that the activated carrot fiber can increase stability and in particular contribute to turbidity stabilization. In addition, the carrot fiber can here increase viscosity, act as a good emulsifier and help to improve flavor release.

The use of the activated carrot fiber in milk substitution products and milk products can provide the following advantages: increased stability, turbidity stabilization, better emulsification, better mouthfeel, texturing, reduction of nutritional value, increased creaminess, substitution of emulsifying salts, reduction of syneresis, improved spreadability and fat substitution.

Selected milk substitution products or milk products are, for example, desserts, yoghurt, yoghurt drink, non-fermented products, fermented drinks, fermented products, processed cheese, cream cheese products.

The use of the activated carrot fiber in ice cream or frozen desserts can provide the following advantages: retardation of crystal growth, form stability in case of heating, improvement of melting behavior, fat substitution, increased creaminess, better mouthfeel, optimization of nutritional value, improved flavor release.

The ice cream or frozen dessert may contain alcohol or not, be fat-free or high in fat content; it may contain insect protein, milk or milk components or even, as vegan ice cream, be free of animal protein. The ice cream or frozen dessert may also be fruit-and/or vegetable-based.

The use of the activated carrot fiber in sweets and especially in chewing gum articles can provide the following advantages: improved abrasive behavior, water retention and improved flavor release.

The use of the activated carrot fiber in sweets and especially in chocolate products can provide the following advantages: fat substitution, processing aid, process stability, better emulsification and thus reduction of grease leak, better viscosity, texturing, optimization of nutritional value (e. g. by reduction in sugar).

The use of the activated carrot fiber in sweets and especially in jelly-type products can provide the following advantages: texturing, improved gelling, adaptation of viscosity, process optimization, reduction in stickiness and better processing.

Some examples of respective sweets are: fruit pieces, jelly products with different Brix contents, jelly products containing fruit, jelly products containing vegetables, these jelly products in combination with nuts or nut derivatives, and sweet confectionary fillings.

The use of the activated carrot fiber in fruit- and/or vegetable-containing drinks potentially containing additional products such as cereals, nuts etc. can provide the following advantages:

increased stability, turbidity stability, good emulsification of juices, better mouthfeel, texturing and reduction in nutritional value.

The fruit- and/or vegetable-containing drink can comprise a large range in terms of viscosity, ranging from thin-flowing to spoonable. In addition to sugary drinks, drinks which are reduced in sugar, sugar-free or salty can also be used. Smoothies are preferred.

The use of the activated carrot fiber in bake-stable fillings can provide the following advantages: form stability, reduction in syneresis, easy introduction, enhanced processing. Advantageously, the carrot fiber can be employed for fillings with a low Brix content of 30-45% dry matter or even lower.

The bake-stable fillings can be fillings containing fruit, vegetables, chocolate, nuts, cereals, cheese or any combination thereof.

The use of the activated carrot fiber in deep-frozen products, and in particular deep-frozen bakery products, can provide the following advantages: enhanced stability of deep-frozen bakery products in terms of loss of volume over storage time, network stabilization, support of gelling in the bakery product and support of gluten network stability.

The use of the activated carrot fiber in baked goods can provide the following advantages: improved elasticity of dough, prolonged freshness, delayed retrogradation, reduction of surface stickiness, improved machine runability (e. g. in case of rye and spelt), optimization of break stability, maintaining of crispness, enhancement of yield of dough and reduction of pastry loss.

The use of the activated carrot fiber in sprinkled baked goods can provide optimized adhesion to e. g. cereals, spices or the like. This applies to frozen and non-frozen products.

The use of the activated carrot fiber in gluten-free baked goods can provide the following advantages: improved elasticity of dough, prolonged freshness, delayed retrogradation, reduction of surface stickiness, improved machine runability, optimization of break stability, maintaining of crispness, enhancement of yield of dough, reduction of pastry loss. The activated carrot fiber here provides a substantial contribution to viscosity build-up. It also supports the starch network.

The use of the activated carrot fiber in extrudates can provide the following advantages: support of extrudability, improved volume result, fine pore structure. This applies to a broad spectrum of extruded products, such as e. g. cereal, fruit, vegetable, protein or meat extrudates.

The use of the activated carrot fiber in meat substitutes based on plant proteins can provide the following advantages: enhanced form stability, enhanced water retention, enhanced emulsification, advantageous texturing, bite optimization, stabilization of matrix, improved cohesion.

The use of the activated carrot fiber in savory products can provide the following advantages: reduction of syneresis, advantageous texturing, stabilization, easy introduction, good form stability, maintaining/support of the typical structure.

The use of the activated carrot fiber in soups or sauces can provide the following advantages: spillover protection due to gelation at respective temperatures, melting at respective temperatures, optimum gelling; better mouthfeel, good emulsification, stabilization, advantageous texturing.

The use of the activated carrot fiber in products based on insects or insect proteins can provide the following advantages: enhanced form stability, enhanced water retention, enhanced emulsification, advantageous texturing, bite optimization, stabilization of matrix, improved cohesion.

The use of the activated carrot fiber in meat and sausage products can provide the following advantages: reduction or substitution of added salts (e.g. phosphates), increased water binding, enhanced emulsification, optimization of cutting properties, enhancement of elasticity, increased water retention, delayed drying on the surface, fat substitution, optimization of nutritional value (e. g. by fat or salt reduction).

The use of the activated carrot fiber in products containing alcohol can provide the following advantages: stabilization at alcohol contents to be defined, good viscosity adjustment, enhanced emulsification, good water binding, better mouthfeel and increased creaminess. These products can comprise a broad spectrum, from spirits such as liqueurs and jellies containing alcohol down to fillings containing alcohol.

The use of the activated carrot fiber in instant products can provide the following advantages: good carrier substance or good separating agent between the functional components, good viscosity build-up in cold or hot media, enhanced emulsification, advantageous texturing, stabilization and good dispersibility.

The use of the activated carrot fiber in artificial, i. e. in particular plant-based casings, can provide the following advantages: softer casings, optimized elasticity, good coating of the casings. Here, a combination with pectin is advantageous.

The use of the activated carrot fiber in dietary foods and in particular enteral feeding can provide the following advantages: good viscosity and ductility, easy swallowing of the food, homogeneous distribution of the agents contained.

The use of the activated carrot fiber in nutritional supplements can provide the following advantages: good viscosity, increase in dietary fiber content, stabilization, advantageous mouthfeel, fat substitute, good texturing, good emulsification.

The activated carrot fiber used according to the invention can be used as a foaming agent or whipping agent for foam stabilization. The possible advantages are: increased stability, enhanced formation and stability of emulsions, better mouthfeel, texturing, reduction in nutritional value, increased creaminess, enhanced spreadability, fat substitute, optimized destabilization of fat agglomerates.

Selected products for this type of use are foamed desserts (milk-or non-milk-based), cream, Froop® (creamy yoghurt with top layer of fruit puree) and ice cream.

The activated carrot fiber used according to the invention can be employed as an emulsifier. The possible advantages include: improved gloss, better mouthfeel, fat substitute, increased creaminess, no overemulsification, better formation and stability of emulsions, optimization of nutritional value, texturing, stabilization and optimization of yield point. The carrot fiber can here be used for emulsions with a great variety of fat contents: from fat-free emulsions to up to 80% of fat content.

The activated carrot fiber used according to the invention can be used as a carrier substance. For instance, it can be a carrier of active agents, flavors or colors.

The activated carrot fiber used according to the invention can be employed as a separating agent or free flow enhancer. It forms a protective layer between hygroscopic surfaces. Here, ease of use is advantageous.

The activated carrot fiber used according to the invention can be employed for manufacturing textile fibers and thus, textiles.

In one embodiment, the activated carrot fiber can be used for producing feedstuff. The person skilled in the art can employ all types of feedstuff known to him as products. Advantageously, the feedstuff is selected from the group consisting of feedstuff rich in starches, oleaginous feedstuff, feedstuff rich in protein, extrudate feedstuff, wet feed, binder, bird feedstuff rod, rodent feedstuff rod, fish bait, supplement feedstuff, feedstuff for particular nutritional purposes and dietary feedstuff.

The use of the activated carrot fiber in feedstuff in the form of wet feed can provide the following advantages: good texturing and structuring, good emulsification, stabilization, enhanced flavor release and optimization of nutritional value.

The use of the activated carrot fiber in feedstuff in the form of extrudates can provide the following advantages: finer pore structure and better volume result.

In one embodiment, the activated carrot fiber can be used for the production of animal supplies. The person skilled in the art can use all types of animal supplies known to him as products. Advantageously, the animal supply is an animal bedding.

The use of the activated carrot fiber in animal bedding can provide the following advantages: high water absorption capacity and good retention.

In one embodiment, the activated carrot fiber can be employed for producing a hygiene article. Here, the person skilled in the art can use all hygiene articles known to him as products. Advantageously, the hygiene article is selected from the group consisting of wet wipes, diapers, incontinence articles such as protective trousers or incontinence pants, sanitary towels, tampons, panty liners and softcups.

The use of the activated carrot fiber in products such as wet wipes can result in good water binding and good water retention capacity.

In one embodiment, the activated carrot fiber can be employed for producing a personal care product. The person skilled in the art can use all personal care products known to him as products. Advantageously, the personal care product is selected from the group consisting of soap, shower gel, bath additives, skin creams, lotions, gel, sunscreen, sun cream, repellent, shaving cream, shaving soap, epilator cream, toothpaste, dentition adhesive medium, shampoo, hair forming agents, hair-setting products, hair colorants, facial make-up, eye care products, lip care products, nail polish and self-tanning agents.

The use of the activated carrot fiber in products such as toothpaste, dentition adhesive medium or casting compounds can provide the following advantages: good abrasiveness, good adhesion, smooth and soft mouthfeel, good emulsification, targeted viscosity formation, stabilization, control of gelling speed.

The use of the activated carrot fiber in products such as shampoos or creams can result in vitalization, moisture-stabilizing effect on the skin (delayed drying out) and good skin tolerability.

The use of the activated carrot fiber in liquid-absorbing products such as diapers, incontinence articles like protective trousers or incontinence pants, sanitary towels, tampons, panty liners and softcups, can provide the following advantages: high water absorption capacity and good retention.

In one embodiment, the activated carrot fiber can be used for producing a cleaning agent. Here, the person skilled in the art can use all cleaning agents known to him as products. Advantageously, the cleaning agent is selected from the group consisting of detergent, bile soap, washing-up liquid, dishwasher detergent, rinsing agent, neutral cleaner, abrasive cleaner, window cleaner, lime remover, drain cleaner, brake cleaner, alcohol cleaner, all-purpose cleaner, glass cleaner, sanitary cleaner, toilet cleaner, toilet gel, toilet soap, carpet cleaner, car care material, oven cleaner, bathroom cleaner and metal polish, shoe polish, oil absorbing and anti-dust agent.

The use of the activated carrot fiber in detergents can provide the following advantages: good adhesion to the toilet wall, good and stable gelling, advantageous abrasiveness, good solubility.

The use of the activated carrot fiber in toilet gels or toilet soaps can provide the following advantages: as a separating agent, a good separation of the functional components and homogeneous distribution of the abrasive components and agents.

The use of the activated carrot fiber in liquid detergents, and in particular in washing-up liquids, can provide the following advantages: as a separating agent, a good separation of the functional components and homogeneous distribution of the abrasive components and agents; good emulsification.

The use of the activated carrot fiber in shoe polish can provide the following advantages: good and stable emulsification, advantageous texturing.

In one embodiment, the activated carrot fiber can be employed for producing a coating agent. The person skilled in the art can use all coating agents known to him as products. Advantageously, the coating agent is selected from the group consisting of antistatic coating, oil-repellent coating and anti-block coating.

In one embodiment, the activated carrot fiber can be employed for producing an explosive. The person skilled in the art can use all explosives known to him as products. Advantageously, the explosive is a gelatinous explosive.

The activated carrot fiber can be employed as a separating agent in the explosive. It can reduce hygroscopicity, control gelation and facilitate processing.

In one embodiment, the activated carrot fiber can be employed for producing a lubricant. The person skilled in the art can use all lubricants known to him as products. Advantageously, the lubricant is selected from the group consisting of liquid lubricants, such as lubricating oil and cooling lubricant, lubricating grease and solid lubricant.

The use of the activated carrot fiber in a lubricant can provide the following advantages: targeted control of viscosity and yield point, stabilization of the emulsion.

The use of the activated carrot fiber in a coolant can provide the following advantages: targeted control of viscosity and yield point, and thus optimized energy absorption for improving cooling capacity.

In one embodiment, the activated carrot fiber can be employed for producing a plastic product. The person skilled in the art can use all plastic products known to him as products. Advantageously, the plastic product is a carrot fiber-reinforced plastic or a wood-plastic composite (WPC).

Production of an alternative plastic product is advantageously performed by production of a compacted product. In this manner, flowerpots, straws or pallets, for example, can be produced.

In one embodiment, the activated carrot fiber can be employed for production of a varnish. The person skilled in the art can use all varnishes known to him as products. Advantageously, the varnish is selected from the group consisting of alkyd resin varnish, oil varnish, cellulose nitrate varnish, bitumen varnish, tar-containing varnish, phenolic resin varnish, urea resin varnish, melamine resin varnish, polyester varnish, epoxy resin varnish, polyurethane resin varnish, acrylic varnish and powder varnish.

In one embodiment, the activated carrot fiber can be employed for production of a coating agent. Here, the person skilled in the art can use all coating agents known to him as products. Advantageously, the coating agent is selected from the group consisting of glaze, oil paint, dispersion paint, chalk paint, silicate paint and liquid plaster.

The use of the activated carrot fiber in coating agents can provide the following advantages: targeted adjustment of viscosity, good emulsion stabilization and adjustment of yield point, better material adhesion, enhanced processability, e.g. in terms of spreadability or sprayability.

In one embodiment, the activated carrot fiber can be employed for production of a construction material. The person skilled in the art can use all construction materials known to him as products. Advantageously, the construction material is selected from the group consisting of building foam, sound proofing material, insulation material, concrete, screed, mortar, cement, chemical bonded anchors, chemical anchor bolts, asphalt and whisper asphalt.

The addition of the activated carrot fiber to an asphalt mixture results in the formation of a low-noise “whisper asphalt”.

The addition of the activated carrot fiber to construction materials such as concrete, screed, mortar or cement of an asphalt mixture can lead to controlled drying, reduction in the formation of cracks, optimized long-time durability and control of hardening.

The addition of the activated carrot fiber to a sound proofing material or an insulation material can stabilize the matrix and reduce the transmission of heat and sound.

In case of a building foam, the activated carrot fiber can stabilize the foam, thus advantageously influencing the matrix structure.

In one embodiment, the activated carrot fiber can be employed for producing an adhesive. The person skilled in the art can use all adhesives known to him as products. Advantageously, the adhesive is selected from the group consisting of dispersion adhesive, melt adhesive, plastisol, cyanoacrylate adhesive, methyl methacrylate adhesive, unsaturated polyester adhesive, epoxy adhesive, polyurethane adhesive, silicones, phenolic resin adhesive, polyimide adhesive, polysulfide adhesive, bismaleimide adhesive, adhesive based on silane-modified polymers, silicone adhesive and paste.

In adhesives, and in particular in paste, the activated carrot fiber can help to control viscosity in a targeted manner and to improve spreadability.

In one embodiment, the activated carrot fiber can be used for production of a medical product. The person skilled in the art can use all medical products known to him as products. Advantageously, the medical product is selected from the group consisting of powder, juice, lotion, ointment, cream, gel, tablet and rubber article.

The use of the activated carrot fiber in ointments can provide the following advantages: good control of viscosity, good formability, easy swallowing, increased creaminess, homogeneous distribution of agents, good drying, increased stabilization, good emulsification and good skin compatibility.

In one embodiment, the activated carrot fiber can be used for production of a medical product. The person skilled in the art can use all medical products known to him as products. Advantageously, the medical product is selected from the group consisting of wound dressing, emergency bandage, transdermal patch, stoma product and dental casting compound.

The use of the activated carrot fiber in patches can provide the following advantages: good gelling and water absorption with retention of the absorbed liquid. This results in moisture-stabilizing patches.

The use of the activated carrot fiber in stoma products, such as colostomy bags, can provide the following advantages: good water absorption and water binding with retention of the absorbed liquid, good skin compatibility.

In one embodiment, the activated carrot fiber can be used for production of a battery. The person skilled in the art can use all batteries known to him as products. Advantageously, the battery is selected from the group consisting of primary cell, accumulator and solid cell battery.

In one embodiment, the activated carrot fiber can be employed in the area of construction. Advantageously, the use in road and path construction, masonry construction, concrete construction and reinforced concrete construction is comprised.

In one embodiment, the activated carrot fiber can be employed in extraction by drilling boreholes. Usage as addition to drilling fluid or fracfluid is advantageous.

The use of the activated carrot fiber in a drilling fluid or a fracfluid can provide the following advantages: increased viscosity in “drilling mud” or similar drilling liquids, replacement of the oil by a medium with higher viscosity, targeted adjustment of viscosity, oil binding, good emulsification. As a result, the activated carrot fiber can thus be used as an extraction aid in the mining industry.

In one embodiment, the activated carrot fiber can be employed in agriculture. The usage in fertilizers, humectants, soil conditioners, plant substrates, flowerpots or substrate-tablet extrudates is advantageous.

In one embodiment, the activated carrot fiber can be employed for producing a fertilizer. The person skilled in the art can use all fertilizers known to him as products. Advantageously, the fertilizer is a binder for fertilizer cones.

The use of the activated carrot fiber in the production of fertilizers can help to keep the agents suspended and to control viscosity and yield point in a targeted manner.

In a substrate-tablet extrudate, the activated carrot fiber can serve as a carrier and/or a separating agent. The pectin can be detached from the fibers and release the nutrients in an ordered manner; and it can also support moisturization.

In one embodiment, the activated carrot fiber can be employed as a reinforcing agent for producing a composite material. The person skilled in the art can use all composite materials known to him as products. Advantageously, the activated carrot fiber is employed for targeted control of abrasive properties, here as a substitute for microplastics.

As an alternative, the activated carrot fiber can be used for surface treatment of the composite materials.

The use of the activated carrot fiber in the production of a composite material can optimize durability and lead to improved elasticity.

In a further aspect, the invention relates to a product selected from the group consisting of food products, feedstuff, commodity goods, animal need, hygiene products, personal care products, cleaning agents, coating agents, care agents, explosives, lubricants, cooling agents, plastic products, fabrics, imitation leather, varnish, ink, paints, building materials, composite materials, paper, cardboard, adhesives, fertilizers, drugs, medical products, batteries, wherein the product comprises the activated carrot fiber used according to the invention.

In one embodiment, the product contains a portion of between 0.05 wt % and 90 wt o, preferably between 0.1 and 50 wt o, particularly preferably from 0.1 to 25 wt % and especially preferably between 0.5 and 10 wt %, of the activated carrot fiber. For instance, the portion of the activated carrot fiber may be 1.0, 1.25, 1.5, 1.75, 2.0, 2.25, 2.5, 2.75, 3.0, 3.25, 3.5, 3.75, 4.0, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88 or 89 wt %.

### DEFINITIONS

A “carrot fiber” in the sense of the application is a component consisting mainly of fibers, which is isolated from a non-lignified cellular wall of a carrot and consists mainly of cellulose. In a sense, the term “fiber” is a misnomer since macroscopically, the carrot fibers do not appear as fibers but as a powdery product. Other components of the carrot fiber are, among others, hemicellulose and pectin.

An activated carrot fiber according to the present invention is defined by the yield point of the fiber in a 2.5% dispersion or by the viscosity. An activated carrot fiber is thus characterized by a yield point I (rotation) of between 15 and 30 Pa, a yield point I (cross-over) of between 20 and 35 Pa or a viscosity of 800 to 5000 mPas at a shear rate of τ=50 1/s.

Within the context of the invention, a “fatty cream” is understood to be a cream containing cooking oil and/or cooking fat. Cooking fats and cooking oil are lipids suitable for human consumption and having a neutral or characteristic smell and flavor. The substances are either called “cooking fat” or “cooking oil”, depending on whether they are solid or liquid at room temperature.

The expression “bake-stable” according to the invention is used to indicate the fact that if subjected to dry heat, a preparation only expands by a minimal amount (i. e. by maximally 25%), as can be determined by the following baking test method. For this purpose, a preparation is used which before the test, in the cooled state, has a creamy and paste-like consistency, such as a chocolate cream, a fruit preparation or a vegetable preparation. A metal ring of 1 cm height and 60 mm in diameter is placed on a piece of filter paper (Hahnenmühle company, Dassel Germany, type 589/1, DP 5891 090, ø90 mm) and is filled with the preparation to be tested, which is placed on the filter paper and smoothed out on the surface of the metal ring. After evenly drawing off the metal ring, the filter paper, on which the preparation has been spread, is placed on a baking tray and baked in the preheated oven (top and bottom heat) for 10 minutes at 200° C. The form stability (diameter before baking in relation to the diameter after baking) of the preparation is assessed. The diameter of the preparation after baking may at most be 125% of the diameter before baking.

A pectin according to the application is defined as a plant polysaccharide which, as a polyuronide, substantially consists of α-1,4-glycosidically bonded D-galacturonic acid units. The galacturonic acid units are partially esterified with methanol. The degree of esterification describes the portion of carboxylic groups in the galacturonic acid units of the pectin which are present in esterified form, e. g. as methyl esters.

A high methoxyl pectin according to the invention is a pectin with an esterification degree of at least 50%. A methoxyl pectin, in contrast, has an esterification degree of less than 50%. The degree of esterification describes the percentage degree of carboxylic groups in the galacturonic acid units of the pectin which are present in esterified form, e. g. as methyl esters. The degree of esterification can be determined by the method according to JECFA (monograph 19-2016, Joint FAO/WHO Expert Committee on Food Additives).

An “instant product” in the sense of the present invention is defined to be a semi-finished food product generally consisting of powder, granulate or dried ingredients, which are stirred into a cold or hot liquid. Cooking during preparation is omitted.

The term “seafood” in the present application is intended to indicate all edible marine animals which are no vertebrates. Typical seafood are clams and aquatic snails, squids and octopi, shrimps, crabs, crayfish and lobster. Seafood can be caught or be a farmed product.

An “extruder product” (synonymous with extrusion product) according to the invention is a product produced by extrusion, which is in general crispy and/or expanded and which can assume any desired shape depending on the pressing nozzles used in the extrusion process. Examples of extruder products are: snacks such as peanut flakes, breakfast cereals, flatbreads, pasta products, sweets such as marshmallows and various extruded soy products which are used in numerous industrially produced foods, both as individual products and as components.

A “smoothie” is a cold mixed drink consisting of fruit and optional milk products, which is freshly prepared or sold as a finished product. Other than in fruit juices, the entire fruit and partly also the peel are used in smoothies. The basis of smoothies is therefore the fruit pulp or fruit puree which, depending on the recipe, is mixed with juices, water, milk, milk products or coconut milk so as to obtain a creamy and smooth consistency.

Within the context of the invention, a “nutritional supplement” is defined to be a food whose purpose it is to supplement the general diet; which is a concentrate of nutrients or other substances with a dietary or physiological effect, by itself or in combination; and which is marketed in dosed form, especially as capsules, lozenges, tablets, pills, effervescent or other similar forms of administration, powder bags, drop-dispensing bottles, bottles with dropper insert and other similar administration forms of liquids and powders for dosage in small measured amounts.

A “functional food” within the context of the invention is characterized by not only having a nutritional value and providing flavor experience, but in addition serving the purpose of long-term promotion and maintenance of health as a “functional” component. Thus, functional foods are predominantly used for preventive healthcare, improving health status and well-being. Important targets of functional foods are the gastrointestinal tract, the cardiovascular system, skin and the brain. Functional foods are consumed in a normal manner and not as tablets, capsules or powders (like nutritional supplements). The biologically active ingredients of functional foods are called nutraceuticals, which term is to designate their health-promoting effects. Frequently, probiotics and prebiotics, phytochemicals, omega-3 fatty acids, vitamins and dietary fibers are added to functional foods as nutraceuticals.

In the context of the invention and in accordance with the German dietary regulation, a “dietary food” is defined to be a food destined for a specific group of people and for a specific nutritional purpose; in addition, it significantly differs from food for general consumption. They are not used for the general diet of average consumers but for a well-defined group of people, such as people with disorders of digestion, resorption and metabolism, people in “particular physiological conditions” or healthy babies and infants.

The following groups of foods are examples of dietary foods: infant formulae and follow-on formulae; other foods for babies and infants (supplementary diet); low-calorie foods for weight loss; foods for special medical purposes (balanced diets); low-sodium foods including dietary salts which have a low sodium content or are entirely free of sodium; gluten-free foods (without additives); foods for intense muscle training, above all, for athletes; food for people suffering from disorders of the glucose metabolism (diabetics), enteral feeding and liquid food.

“Enteral food” according to the invention is a food which is liquid and of a viscosity low enough so that it can be administered via a feeding tube. It is a completely balanced diet for enteral feeding which is applied via a tube and an application system by the force of gravity or via a pump system. The standard foods cover all requirements of humans for carbohydrates, fats, protein, vitamins and trace elements, additionally containing dietary fibers. An isocaloric standard food has approximately 1.0 to 1.2 kcal/ml with a water content of 80% to 85%. If the energy density is higher, the diet involves high caloric standard food with a lower water content of 64% to 77%, which must be taken into account for liquid balancing.

“Liquid food” within the context of the present invention is a specially composed high-energy food in liquid form which can be drunk. It is used for additional or complete feeding if the patient cannot consume solid foods or can do it only to an insufficient degree.

“Feedstuff” (also briefly called feed) according to the invention is a collective term for all types of animal feed. The term comprises the food for all animals kept by humans, such as livestock, zoo animals, animals for sports or pets. Feedstuff today is specifically formulated for the respective species and purpose. Some examples are: feedstuff rich in starches which is produced from grains, seeds and tubers rich in starches; oleaginous feedstuff, feedstuff rich in protein, that is, containing 35-65% of protein, and other feedstuff obtained either from nature (e. g. fishmeal) or as an after product from industrial production. These are, for instance, bran (from the mill), stillage (production of alcohol), brewer grains (production of beer), pomace (production of wine and juice), molasses and beet pulp from the sugar industry, and other food residues.

“Animal bedding” according to the invention designates materials which are used in animal husbandry for covering the ground in stables and cages and absorb the animals' excrements.

A “wound dressing” is a dressing placed on external wounds in order to prevent the entry of foreign bodies into the wound and absorb blood and wound exudate. In addition, wound dressings may guarantee a warm and humid wound climate promoting healing, alleviate pain by means of contained substances, promote wound healing or have antimicrobial effects.

A “commodity good” in the sense of the present application is an article which, in accordance with § 2 subparagraph 6 of the German Lebensmittel-, Bedarfsgegenstände- and Futtermittelgesetzbuch (LFGB; Code on food, commodity goods and feedstuff), is selected from the list consisting of:
no. 1 materials and objects designed to come in contact with food (also called food commodity goods),
no. 2 packaging, containers or other casings designed to come in contact with cosmetics,
no.3 objects designed to come in contact with the mucosae of the mouth,
no. 4 objects designed for hygiene,
no. 5 toys and joke articles,
no. 6 objects designed to come not only temporarily in contact with the human body, such as clothing, bed cloth, masks, wigs, hairpieces, artificial eyelashes, wristbands,
no. 7 cleaning and care articles designed for domestic use or for commodity goods in the sense of number 1,
no. 8 impregnation agents and other equipment for commodity goods in the sense of number 6 which are designed for domestic use,
no. 9 means and objects for the improvement of smell in rooms designed for the accommodation of people.

A “filtering aid” according to the invention is a chemically inert substance which physically-mechanically supports filtration. It is not supposed to be confused with or equated to a flocculant. Filtering aids are employed to facilitate cleaning of the actual filter or filter insert or to prevent solid substances in the suspension from clogging the filter or getting into the filtrate. Filtering aids are generally used in water treatment, filtration of drinks and more specifically in the chemical industry.

An “egg substitute” according to the invention is a plant-based food which optically or in terms of taste as well as in its properties in the preparation of dishes resembles the whole egg, the egg white or the yolk. Use of a plant-based egg substitute can be associated with easier handling, a lower price and a lesser risk of food poisoning.

A “glazing agent” according to the invention is a food additive which protects the food from losses in smell, taste and moisture, promotes gloss or prolongs freshness. It can also function as a separating agent.

A “humectant” according to the invention is a food additive which prevents the drying out of foods by binding water added during production (i. e. preventing evaporation) or by attracting humidity of the air during storage. By preventing hardening of the finished food, it acts as a softening agent. In sweets, it counteracts crystallization of the sugar.

A “dietary fiber” according to the invention is a largely indigestible food component, generally consisting of carbohydrates, which predominantly exists in plant-based foods. For purposes of simplicity, dietary fibers are classified into water-soluble fibers (like pectin) and water-insoluble fibers (such as cellulose). Dietary fibers are considered to be an important part of the human diet. The EU Regulation on nutrition labelling globally assigns them a calorific value of 8 kJ/g.

A “reinforcing agent” according to the present invention is a single substance of a composite material. As the name suggests, the reinforcing agent is to guarantee rigidity and stiffness of the composite. What is most significant, in addition to its type, is the form of the reinforcing agent, namely whether it is present as a particle, a fiber or in layers. In particular, by “reinforcing agents” (“reinforcement”) the organic additives employed in plastics are understood which reinforce the plastics matrix. By reinforcement, the enhancement of mechanical and physical properties, such as elasticity, bending strength, creep mechanisms and heat deflection temperature are understood. Reinforcing agents are employed in a targeted manner to improve these material properties.

“Gelling agents” according to the invention are food additives which swell in water or bind water, i. e. lead to gelling. They form a gelatinous mass and give a viscid or solid consistency to soups, sauces or pudding.

A “solidifier” according to the invention is a food additive which ensures that solidity and freshness of a food remain during and after processing. It reacts with various components, such as e. g. pectin. A solidifier can be, for instance, a calcium salt which reacts with an ingredient of the product, such as the pectin in fruit.

A “texturant” according to the present application is a substance with the ability to provide a special texture to a product. By “texture”, in this context, the surface properties of food are understood which can be perceived by the tactile sense, in particular the mouthfeel of a product.

A “thickener” according to the present application is a substance which is, above all, capable of binding water. By removing unbonded water, viscosity is increased. Above a certain concentration, which is characteristic for each individual thickener, network effects are additionally produced which lead to a generally disproportionate increase in viscosity. Thickeners are capable of providing a product with a specific consistency. Thickening in this context means an increased viscosity or solidity of the product as a result of employment of the thickener.

A “filler” according to the invention is an insoluble additive which, added in large amounts to the basic material (the matrix), strongly alters, among others, the mechanical, electric and processing properties of materials and can at the same time significantly decrease the portion of the matrix, which is typically more expensive, in the finished product. Preferably, a filler is a food additive which forms part of the food volume without substantially contributing to its content of usable energy. In this manner, the actual energy content per volume or per mass of the foods is reduced.

A “carrier” according to the invention is a substance to which other substances can be attached (physically bonded), i. e. which can “carry” other substances. For instance, a pharmaceutical agent or an aroma ingredient, which is otherwise difficult to dose, can be bonded to a carrier which is easier to dose. Preferably, the carrier is a technical adjuvant in the food industry and can transport flavors into the products, with the appearance and taste of a food generally not being modified by the carrier itself. As technical adjuvants, they do not have to be listed in the list of ingredients since they in themselves do not cause effects in the final product.

Within the context of the present invention, an “emulsifier” is an adjuvant which is used to combine two fluids which by themselves do not mix, such as oil and water, to form a finely distributed mixture, called an emulsion, and to stabilize it. The same applies to the mixing of solid, non-soluble substances with a liquid in order to stabilize a suspension. Preferably, the emulsifier is a food additive.

A “separating agent” according to the invention is a food additive or technical adjuvant which prevents sticking together or agglomeration of foods. Thus, separating agents are also among the agents which increase or maintain flowability. Thus, separating agents prevent salt, for example, from turning lumpy and individual pieces of candy from sticking together and forming one single block of sugar. As technical adjuvants, they are employed in industrial processing and production of foods. Technical adjuvants are food additives which are added in order to facilitate technical processes, such as cutting and filtration. In the final product, however, the technical adjuvants may not be present at all or only in low residual amounts which are unavoidable.

A “free flow agent” according to the invention is a separating agent which is added to crystalline substances in order to prevent agglomeration of the individual crystals, mainly for the purpose of better machine processability. Its use is to prevent the lumping of, for instance, table salt before or during processing, which makes it more difficult to dose.

A “stabilizer” according to the invention is a food additive which has the property of maintaining, if it is added to a metastable system, the characteristics, manageability, flavor or other parameters of this system in a defined manner, and thus of stabilizing it. A stabilizer can serve one or more functions.

An “improving agent for baking stability” according to the invention is characterized in that a liquid, viscous or cream-like composition, to which the improving agent is added, only spreads or flows minimally after the agent has been added and dry heat is applied.

A “foaming agent” according to the invention is a food additive which ensures that a homogeneous dispersion of gas forms in liquid or solid foods. Thus, foaming agents ensure that gases distribute evenly in liquids or solids.

A “whipping agent” according to the invention is a food additive which, after it has been added to a mass, allows increasing the volume of the mass by whipping air into it. Whipping agents stabilize the mass and thus facilitate the handling thereof. Whipping agents are used in the food industry for instance to produce sponge cake, chocolate mousse and other desserts.

An emergency bandage, also colloquially called an adhesive plaster or patch, is a piece of wound dressing attached to an adhesive tape. It is used to cover small wounds.

A “transdermal patch” according to the invention is a dosage form for the systemic administration of drugs in the form of patches. It is attached to the skin and releases the agent in a controlled manner, which agent is then resorbed by the skin. The agent reaches the circulatory system without previously having been broken down in the gastrointestinal tract or the liver.

Within the framework of the application, a “stoma” is understood to be an artificial connection between a body cavity and the body surface. Typical examples of a stoma are colostomy, ileostomy and urostomy. For the receiving of excrements, such as stool and/or urine, stoma products (e. g. ileostomy bags) are employed. These are bags attached to an adhesive surface. The adhesive surface is attached to the abdomen around the stoma and adheres to the skin.

Within the framework of the application, “cleaning agents” are consumables which are used to clean various articles and objects. They cause or support the removal of impurities due to usage, or of residues and adhering substances from the manufacturing process of the object. Different fields of application require different cleaning agents. For laundry and textiles, detergents (heavy-duty detergents, color detergents, fabric softeners etc.) or bile soap are employed. For dishes (cookware, dinnerware and cutlery) washing-up liquid, dishwashing agents or rinsing aids are used. For surfaces in living spaces and offices: neutral cleaners, abrasive cleaners (scouring powder) or window cleaners. Other cleaning agents are, for instance, lime removers, drain cleaners, brake cleaners, alcohol cleaners, all-purpose cleaners, glass cleaners, sanitary cleaners, toilet cleaners, carpet cleaners, car care material, oven cleaners, bathroom cleaners and metal polish.

In the context of the present application, a “lubricant” is a substance used for lubrication which serves to reduce friction and wear as well as to provide cooling, vibration damping, sealing and protection against corrosion. Principally, all lubricants consist of a basic liquid (generally base oil) and of other ingredients called additives. Examples of lubricants are liquid lubricants (lubricating oils and cooling lubricants), lubricating greases and solid lubricants (such as graphite).

Within the framework of the invention, “coolants” are liquid or solid substances or mixtures of substances which are used for the dissipation of heat.

A “composite material” is a material consisting of two or more bonded substances which has material properties different from those of its individual components. For the properties of composite materials, substantial properties and the geometry of the components are important. Size effects play a particular role.

Within the context of the present invention, “paints” are liquid or pasty, rarely powdery substances of mixtures which, if applied on surfaces, are subjected to physical drying or chemically curing processes. According to DIN 55945, a paint is a liquid or pasty coating agent which is mainly applied by brushing or rolling.

An “adhesive” according to the invention is a non-metal substance which is capable of bonding materials by surface adhesion and its inner stability (cohesion). That is, it is a process material employed to bond different materials in the adhesive bonding method. Examples are dispersion adhesive, melt adhesive, plastisol, cyanoacrylate adhesive, methyl methacrylate adhesive, unsaturated polyester adhesive, epoxy adhesive, polyurethane adhesive, silicones, phenolic resin adhesive, polyimide adhesive, polysulfide adhesive, bismaleimide adhesive, adhesive based on silane-modified polymers, silicone adhesive.

“Drilling fluids” (also called drilling mud) in the context of the present application are liquids which are pumped through the borehole in mining. There are two basic types of drilling fluids; those based on water and those based on oil. Drilling fluids are basically used for stabilizing a borehole, cleaning the borehole bottom and removing the bottom material (cuttings) which have been drilled out. In addition, they dissipate the substantial frictional heat which has been induced on the drill bit, thus cooling and lubricating the drilling tool. In addition, they reduce the frictional resistance of the drill bit and the rotating boring rods and dampen their oscillations.

Fracking is a method of creating, widening and stabilizing cracks in the rocks of a deep deposit with the aim of increasing the permeability of the deposit rocks. In this manner, gases or liquids contained therein can flow more easily and continuously to the bore and be extracted there. In fracking, a fluid (“fracfluid”) is pressed into the geological horizon, from which extraction is supposed to take place, at a high pressure of typically several hundred bar. The fracfluid is water to which in general supporting means, such as quartz sand, and thickeners have been added.

At this point, it is explicitly pointed out that features of the solutions described above, in the Claims and/or in the Figures can also be combined, if desired, in order to achieve cumulated implementation of the explained features, effects and advantages.

All features disclosed in the application documents are claimed as essential for the invention provided that they are, individually or in combination, novel over the state of the art.

It is explicitly pointed out that within the framework of the present patent application, indefinite articles and numerals such as “one”, “two” etc. are normally to be understood as indicating a minimum, i. e. “at least one . . . ”, “at least two . . . ” etc., unless it becomes explicitly clear from the context or is obvious or imperative to the person skilled in the art from a technical point of view that only “exactly one . . . ”, “exactly two . . . ” etc. can be intended.

Other advantages, particularities and expedient embodiments of the invention will become clear from the dependent Claims and the following presentation of preferred embodiments by means of the Figures.

The embodiments shown here are only examples of the present invention and are therefore not to be understood as limiting. Alternative embodiments considered by the person skilled in the art are equally comprised by the scope of protection of the invention.

The plant fiber in the usage according to the invention is, in one embodiment, a de-pectinized plant fiber, preferably a de-pectinized fruit fiber. If the term “de-pectinized fiber” is used here or otherwise in the application, what is intended is that the content of pectin in the fiber has been reduced with respect to the original fiber.

Furthermore, it is pointed out that a fruit fiber according to the invention is a plant fiber according to the above definition which is isolated from a fruit. By “fruit”, what is intended here is the whole of all organs of a plant which originate from a blossom, comprising both classic fruits and fruit vegetables.

### EXAMPLES OF EMBODIMENT

### 1 Preparation of an Activated Carrot Fiber

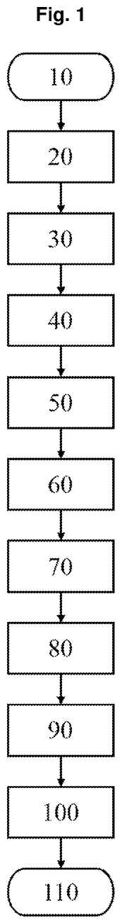

FIG. 1 is a schematic representation of the method of producing the carrot fiber as described before in the form of a flowchart. Starting from the carrot pulp 10, the pulp is subjected to hydration 20. During this process, the dry pulp is rehydrated by incubation in demineralized water at 45° C. over 1 hour and subsequently the hydrated dry mass is separated from the aqueous liquid by means of a decanter. In step 30, the pH value is adjusted. For this purpose, a pH value of 4.0 is set which is optimal for the subsequent enzymatic treatment. This is followed by enzymatic treatment 40 of the rehydrated dry pulp by incubation with a pectin methyl esterase and/or cellulase in an aqueous suspension over 1 to 2 hours at 40 to 70° C. Then, two alcohol washing steps 50 and 70 are performed with subsequent solid-liquid separation by means of decanters 60 and 80. In step 90, finally, the fibers are gently dried by vacuum drying, followed by a milling and sieving step 100 so as to obtain the activated carrot fibers 110 used according to the invention.

### 1.1 Preparation of a 2.5 Wt % Fiber Dispersion

### Formula:

2.5 g fiber

97.5 g demineralized water (room temperature)

duration of sprinkling: 15 seconds

The respective amount of demineralized water (room temperature) is introduced into a 250 ml beaker. The exactly weighed amount of fiber is slowly and directly poured into the stirring maelstrom with the stirring unit (Ultra Turrax) running at 8000 rpm (level 1). The sprinkling duration depends on the amount of fibers; it is to last 15 seconds per 2.5 g of sample. Then the dispersion is stirred for exactly 60 seconds at 8000 rpm (level 1). If the sample is to be used for determining viscosity or for determining the yield point I (rotation), yield point I (cross-over) or the dynamic Weissenberg number, it is placed in a temperature-controlled water bath at 20° C.

For measuring viscosity or for measuring the yield point I (rotation), the yield point I (cross-over) or for measuring the dynamic Weissenberg number, the sample is carefully given, after exactly 1 hour, into the measurement system of the rheometer, and the respective measurement is started. If the sample settles, it is carefully stirred up by means of a spoon directly before bottling.

### 1.2 Preparation of a 2.5 Wt % Fiber Suspension

### Formula:

2.50 g fiber

97.5 g demineralized water (room temperature)

The respective amount of water (room temperature) is introduced into a 250 ml beaker. The exactly weighed amount of fiber is slowly poured in under constant stirring with a plastic spoon. Then the suspension is stirred until all fibers are watered. If the sample is to be used for determining viscosity or for determining the yield point II (rotation), the yield point II (cross-over) or for determining the dynamic Weissenberg number, it is placed in a temperature-controlled water bath at 20° C.

For measuring viscosity or for measuring the yield point II (rotation), the yield point II (cross-over) or for measuring the dynamic Weissenberg number, the sample is carefully filled into the measurement system of the rheometer after exactly 1 hour, and the measurement is started. If the sample settles, it is carefully stirred up by means of a spoon directly before bottling.

### 1.3 Test Method for Determining Water Binding Capacity

### Implementation:

The sample is left to swell over 24 hours with a water excess at room temperature. After centrifugation and subsequent decanting of the supernatant, the water binding capacity can be gravimetrically determined in g H2O/g of sample. The pH value in the suspension is to be measured and documented.

The following parameters are to be observed:

| Weighed portions: | | |
|---|---|---|
| plant fiber: | 1.0 g (in centrifuge tube) | |
| water addition: | 60 | ml |
| centrifugation: | 4000 × g | |
| duration of centrifugation: | 10 | min |

20 minutes after beginning of centrifugation (i. e. 10 minutes after the end of centrifugation), the water supernatant is separated from the welled sample. The sample with the bound water is weighed.

The water binding capacity (WBC) in g H2O/g sample can now be calculated with the following formula:

$$W B C ( \frac{g {H}_{2} O}{g sample} ) = \frac{sample with bound water ( g ) - 1. g}{1. g}$$

### 1.4 Test Method for Determining the Yield Point (Rotational Measurement)

### Measurement Principle:

This yield point is an indicator of the structural strength and is determined by rotational measurement, by increasing the shear stress acting on the sample over time until the sample begins to flow.

Shear stresses below the yield point merely cause an elastic deformation; it is only shear stresses above the yield point that will cause the sample to flow. This value is determined by measuring when a defined minimum shear rate y is exceeded. According to the present method, the yield point τ_{O} [Pa] is exceeded at shear rate γ≥0.1 s⁻¹.

| | |
|---|---|
| measuring device: | Rheometer Physica MCR series |
| | (e.g. MCR 301, MCR 101) |
| measuring system: | Z3 DIN or CC25, respectively |
| measuring vessel: | CC 27 P06 (ribbed measuring vessel) |
| number of measuring sections: | 3 |
| measuring temperature: | 20° C. |

measuring parameters:

### 1^{st} Section (Resting Period):

| | | |
|---|---|---|
| section settings: | default parameter: | shearing stress [Pa] |
| | value: | 0 Pa constant |

| | | |
|---|---|---|
| section duration: | 180 | s |
| temperature: | 20° | C. |

2^{nd} Section (Determining of Yield Point after Rotation Measurement):

| | | |
|---|---|---|
| section settings: | default parameter: | shearing stress [Pa] |
| | profile: | ramp log. |

| | | |
|---|---|---|
| initial value: | 0.1 | Pa |
| final value: | 80 | Pa |
| section duration: | 180 | s |
| temperature: | 20° | C. |

### Evaluation:

The yield point To (unit [Pa]) is read out in section 2 and is the shearing stress (unit: [Pa]) at which the shear rate is for the last time γ≤0.10 s⁻¹.

The yield point measured with the rotation method is also called “yield point (rotation)”.

The yield point (rotation) was measured using a fiber suspension (the fiber was simply stirred in with a spoon=corresponding to one fiber which was not additionally activated) and is also called “yield point II (rotation)” within the context of the invention. The yield point was also measured using a fiber dispersion (stirred in under the effect of high shearing forces, e. g. with Ultra Turrax =corresponding to an additionally activated fiber) and is also called “yield point I (rotation)” within the context of the invention.

### 1.5 Testing Method for Determining Yield Point (Oscillation Measurement)

### Measurement Principle:

This yield point is also an indicator of the structural strength and is determined in an oscillation test by increasing the amplitude at constant frequency until the sample is destroyed due to the ever-increasing excursion and starts to flow.

Below the yield point, the substance behaves like an elastic solid, i. e. the elastic portions (G′) amount to a larger portion than the viscous portions (G″) whereas when the yield point is exceeded, the viscous portions of the sample increase and the elastic portions decrease.

By definition, the yield point is exceeded at the amplitude when the amount of viscous portions equals that of elastic portions; G′=G″ (cross-over); the corresponding shear stress is the respective measured value.

| | |
|---|---|
| Measuring device: | Rheometer Physica MCR series |
| | (e.g. MCR 301, MCR 101) |
| Measuring system: | Z3 DIN or CC25 |
| Measuring vessel: | CC 27 P06 (ribbed measuring vessel) |

### Measuring Parameters:

| | | |
|---|---|---|
| section settings: | amplitude defaults: | deformation [%] |
| | profile: | ramp log. |
| | value: | 0.01-1000% |
| | frequency defaults: | frequency [Hz] |
| | profile: | constant |

| | | |
|---|---|---|
| frequency: | 1.0 | Hz |
| temperature: | 20° | C. |

### Evaluation:

By means of the rheometer software Rheoplus, the shear stress at cross-over is evaluated after the linear viscoelastic range has been exceeded.

The yield point measured with the oscillation method is also called “yield point (cross-over)”.

The yield point (cross-over) was measured using a fiber suspension (the fiber was simply stirred in with a spoon =corresponding to a not additionally activated fiber) and is also termed “yield point II (cross-over)” within the framework of the invention. The yield point was additionally measured using a fiber dispersion (stirred in under the effect of high shearing forces, e. g. with Ultra Turrax =corresponding to an additionally activated fiber) and is also called “yield point I (cross-over)” in the context of the invention.

### Implications:

If the yield point of the suspension of the fibers used according to the invention, stirred in with a spoon (corresponding to a not additionally activated fiber), is compared to that of a fiber dispersion stirred in under high shearing forces, such as Ultra Turrax (corresponding to an additionally activated fiber), a statement on the advantage/necessity of an activation can be made. The measuring results are summarized in the table below. As can be expected, the yield point increases by shear activation in the dispersion. However, also the fiber suspension has a yield point which, with τ_{O}II>1.5 Pa, is sufficiently high to achieve a creamy texture. Therefore, activation of the fiber is not absolutely necessary.

| | rotation | | cross-over | | |
|---|---|---|---|---|---|
| | τₒII[Pa] | τₒI [Pa] | τₒII [Pa] | τₒI [Pa] | |
| fiber | suspension | dispersion | suspension | dispersion | activation |
| activated | 23.3 | 22.5 | 28.6 | 25.3 | not |
| carrot | | | | | absolutely |
| fiber | | | | | necessary |
| according | | | | | |
| to the | | | | | |
| invention | | | | | |

### 1.6 Testing Method for Determining the Dynamic Weissenberg Number

### Measuring Principle and Meaning of the Dynamic WEISSENBERG Number:

The dynamic Weissenberg number W′ (Windhab E, Maier T, Lebensmitteltechnik 1990, 44: 185f) is a derived variable in which the elastic portions (G′) determined in the linear viscoelastic range in the oscillation test are related to the viscous portions (G″):

$${W}^{′} = \frac{{G}^{′} ( ω )}{{G}^{″} ( ω )} = \frac{1}{tan δ}$$

The dynamic Weissenberg number is a variable which correlates particularly well with the sensorial perception of consistency and can be regarded quite independently of the absolute firmness of the sample.

A high value of W′ means that the fibers have a predominantly elastic structure, whereas a low value of W′ indicates structures with clearly viscous portions. The creamy texture typical of fibers is achieved if the W′ values lie within a range of approximately 6-8; if the values are lower, the sample is assessed to be aqueous (thickened less strongly).

### Material and Methods:

| | |
|---|---|
| measuring device: | Rheometer Physica MCR series, |
| | e.g. MCR 301, MCR 101 |
| measuring system: | Z3 DIN or CC25 |
| measuring vessel: | CC 27 P06 (ribbed measuring vessel) |

### Measuring Parameters:

| section settings: | amplitude defaults: | deformation [%] |
|---|---|---|
| | profile: | ramp log |
| | value: | 0.01-1000% |

| | | |
|---|---|---|
| frequency: | 1.0 | Hz |
| temperature: | 20° | C. |

### Evaluation:

The angle of phase difference 5 is read out within the linear viscoelastic range. Subsequently, the dynamic Weissenberg number W′ is calculated with the following formula:

$${W}^{′} = \frac{1}{tan δ}$$

Measuring Results and their Implications:

Comparing the dynamic Weissenberg number W′ for the suspension of a fiber used according to the invention, stirred in with a spoon (corresponding to a not additionally activated fiber) with a fiber dispersion, stirred in with high shearing forces, e. g. Ultra Turrax (corresponding to an additionally activated fiber), a statement on the texture and, in addition, on the necessity of an activation can be made. The measuring results are summarized in the table below. The carrot fiber according to the invention is, with W′ values of 7.9 in the suspension and 8.1 for the dispersion, in the ideal range and thus has an optimum texture. The texture is in both cases creamy. Thus, the results concerning the dynamic Weissenberg number also show that activation of the fiber is not absolutely necessary.

| | W′ | W′ | |
|---|---|---|---|
| fiber | suspension | dispersion | texture |
| activated carrot | 7.9 | 8.1 | creamy with and without |
| fiber according | | | activation, |
| to the invention | | | viscosity/yield point are |
| | | | regulated by dosing |

### 1.7 Testing Method for Determining Firmness

### Method:

150 ml of distilled water are introduced into a beaker. Then, 6.0 g of carrot fibers are stirred into the water with a spoon without formation of lumps. For swelling, this fiber-water mixture is left to stand for 20 min. The suspension is then transferred into a vessel (Ø90 mm). Subsequently, the firmness is measured with the following method:

| | |
|---|---|
| Measuring device: | Texture Analyser TA-XT 2 (company Stable |
| | Micro Systems, Godalming, UK) |

Testing method/option: measuring of force in the direction of pressure/simple test

| | |
|---|---|
| Parameters: | testing speed: 1.0 mm/s |
| | distance: 15.0 mm/s |
| Measuring tools: | P/50 |

According to the present method, the firmness corresponds to the force required by the measuring bob to penetrate into the suspension by 10 mm. This force is then read out from the force-time diagram. It should be mentioned that the unit gram (g) for measured strength has been established from the history of strength measurement.

### 1.8 Testing Method for Determining Particle Size

### Measurement Principle:

In a sieving machine, a set of sieves with a mesh width continuously increasing from the lower sieve to the upper sieve is arranged on top of each other. The sample is placed on the top sieve, i. e. the sieve with the largest mesh width. The sample particles with a diameter larger than the mesh width remain on the sieve; the finer particles fall onto the sieve below it. The portion of sample on the various sieves is weighed and indicated as a percentage.

### Method:

The sample is weighed in to the second decimal digit. The sieves are provided with sieving aids and stacked on top of each other with the mesh width increasing. The sample is quantitatively transferred onto the top sieve; the sieves are clamped in, and the sieving process is performed according to defined parameters. The individual sieves are weighed with the sample and the sieving aid as well as empty with the sieving aid. If for a product, only a limit value within the particle size spectrum is to be tested (e. g. 90% <250 μm), only a sieve with the respective mesh width is used.

### Measuring Defaults:

| sample amount: | 15 | g |
|---|---|---|
| sieving aids: | 2 per sieve bottom | |
| sieving machine: | AS 200 digit, Retsch GmbH company | |
| sieving movement: | three-dimensional | |
| oscillation height: | 1.5 | mm |
| sieving duration: | 15 | min |

The sieve structure consists of the following mesh widths in pm: 1400, 1180, 1000, 710, 500, 355, 300, followed by the bottom.

The particle size is calculated using the following formula:

$$portion per sieve in \% = \frac{final weight in g on the sieve \times 100}{initial sample weight in g}$$

### 1.9 Testing Method for Determining Viscosity

measuring device: Physica MCR series (e. g. MCR 301, MCR 101)

measuring system: Z3 DIN or CC25 (note: the measuring systems

Z3 DIN and CC25 are identical) number of sections: 4

Measuring parameters:

1^{st} section:

| | | |
|---|---|---|
| section settings: | default parameter: | shear rate [s ⁻¹] |
| | profile: | constant |

| | | |
|---|---|---|
| value: | 0 | s⁻¹ |
| section duration: | 60 | s |
| temperature: | 20° | C. |

2^{nd} section:

| | | |
|---|---|---|
| section settings: | default parameter: | shear rate [s⁻¹] |
| | profile: | ramp linear |

| | | |
|---|---|---|
| value: | 0.1-100 | s⁻¹ |
| section duration: | 120 | s |
| temperature: | 20° | C. |

3^{rd} section:

| | | |
|---|---|---|
| section settings: | default parameter: | shear rate [s⁻¹] |
| | profile: | constant |

| | | |
|---|---|---|
| value: | 100 | s⁻¹ |
| section duration: | 10 | s |
| temperature: | 20° | C. |

4^{th} section:

| | | |
|---|---|---|
| section settings: | default parameter: | shear rate [s⁻¹] |
| | profile: | ramp linear |

| | | |
|---|---|---|
| value: | 100-0.1 | s⁻¹ |
| section duration: | 120 | s |
| temperature: | 20° | C. |

### Evaluation:

The viscosity (unit [mPas]) is read out as follows: 4^{th} section at =50 s⁻¹

### 1.10 Testing Method for Determining the Dietary Fiber Content

This method substantially corresponds to the method published by AOAC (Official Method 991.43: Total, Soluble and Insoluble Dietary Fiber in Foods; Enzymatic-Gravimetric Method, MES-TRIS buffer,

First Action 1991, Final Action 1994). The only difference is that here, isopropyl alcohol instead of ethanol is used.

### 1.11 Testing Method for Determining Moisture and Dry Mass

### Measurement Principle:

The moisture content of the sample is understood to be the reduction in mass after drying, determined according to defined preconditions. The moisture content is determined by means of infrared drying with the moisture analyzer Sartorius MA-45 (Sartorius company, Gottingen, Federal Republic of Germany).

### Method:

Approximately 2.5 g of the fiber sample are weighed in on the Sartorius moisture analyzer. The settings of the device can be found in the respective factory measuring instructions. For measuring, the samples are to have approximately room temperature. The moisture content is automatically indicated in percent [%M] by the device. The content of dry substance is automatically indicated in percent [96 S] by the device.

1.12 Testing Method for Determining Color and Lightness

### Measurement Principle:

The measurements of color and lightness are performed with the Minolta Chromameter CR 300 or CR 400, respectively. The spectral characteristics of a sample are determined using standard color values. The color of a sample is described using the hue, the lightness and saturation. With these three basic properties, the color can be represented three-dimensionally.

The hues are located on the external face of the color solid; the lightness changes on the vertical axis and the degree of saturation horizontally. If the L*a*b* measurement system (L-star, a-star, b-star) is employed, L* represents lightness whereas a* and b* indicate both the hue and the saturation. a* and b* indicate the positions on two color axes, with a* being assigned to the red-green axis and b* being assigned to the blue-yellow axis. For indicating the color measurement values, the device converts the standard color values into L*a*b* coordinates.

### Performance of Measurement:

The sample is sprinkled on a white sheet of paper and flattened with a glass plug. For measurement, the measuring head of the chromameter is directly placed on the sample and the trigger is actuated. A triple measurement is performed of each sample and the average value calculated. The L*, a* and b* values are indicated by the device with two decimals.

### LIST OF REFERENCE NUMBERS

10 carrot dry pulp
20 hydration of dry pulp
30 pH adjustment
40 enzymatic treatment with PME and/or cellulase
50 1^{st} washing with alcohol
60 solid-liquid separation decanter
70 2^{nd} washing with alcohol
80 solid-liquid separation decanter
90 vacuum drying
100 milling and sieving step
110 obtained activated carrot fiber

1. Use of an activated carrot fiber for preparing a product selected from the group consisting of food products, feedstuff, commodity goods, animal need, sanitary articles, personal care products, cleaning agents, coating agents, care agents, explosives, lubricants, cooling agents, plastic products, fabrics, imitation leather, varnish, ink, paints, building materials, composite materials, paper, cardboard, adhesives, fertilizers, drugs, medical products, batteries, wherein the activated carrot fiber has a viscosity of 800 mPas to 5000 mPas at a shear rate of τ=50 1/s in a 2.5 wt % aqueous suspension.

2. Use of an activated carrot fiber in the construction area, in extraction by drilling boreholes and in agriculture, wherein the activated carrot fiber has a viscosity of 800 mPas to 5000 mPas at a shear rate of τ=50 1/s in a 2.5 wt % aqueous suspension.

3. Use according to claim 1, wherein the activated carrot fiber in use has one or more of the following functions: as a foaming agent, a whipping agent, a release agent, a free flow agent, a stabilizer, an emulsifier, a carrier, a filler, a texturing agent, a thickener, a gelling agent, a solidifying agent, a dietary fiber, a reinforcing agent, a humectant, a filter aid, an egg substitute, a glazing agent, an improving agent for freeze-thaw stability and an improving agent for baking stability.

4. Use according to claim 1, wherein the activated carrot fiber has one or more of the following properties:
a. a yield point II (rotation) in a 2.5 wt % aqueous fiber suspension of between 15 and 30 Pa, advantageously between 17.5 and 27.5 Pa and particularly advantageously between 20 and 25 Pa;
b. a yield point I (rotation) in a 2.5 wt % aqueous fiber dispersion of between 15 and 30 Pa, advantageously between 17.5 and 27.5 Pa and particularly advantageously between 20 and 25 Pa;
c. a yield point II (cross-over) in a 2.5 wt % aqueous fiber suspension of between 20 and 35 Pa, advantageously between 22.5 and 32.5 Pa and particularly advantageously between 25 and 30 Pa;
d. a yield point I (cross-over) in a 2.5 wt % aqueous fiber dispersion of between 25 and 35 Pa, advantageously between 20 and 30 Pa and particularly advantageously between 22.5 and 27.5 Pa;
e. a dynamic Weissenberg number in a 2.5 wt % aqueous fiber suspension of between 5 and 11 Pa, advantageously between 6 and 10 Pa and particularly advantageously between 7 and 9 Pa;
f. a dynamic Weissenberg number in a 2.5 wt % aqueous fiber dispersion of between 5 and 11 Pa, advantageously between 6 and 10 Pa and particularly advantageously between 7 and 9 Pa;
g. a firmness in a 4 wt % aqueous suspension of between 320 and 510 g, preferably between 350 g and 480 g particularly preferably between 380 and 450 g;
h. a viscosity of 800 to 4800 mPas, preferably 1000 to 4500 mPas and particularly preferably 1200 to 4000 mPas, wherein the activated carrot fiber is dispersed in water as a 2.5 wt % solution and the viscosity is measured with a shear rate of 50 s⁻¹ at 20° C.;
i. a water binding capacity of between 25 and 50 g/g, preferably between 30 and 45 g/g, particularly preferably between 32.5 and 42.5 g/g and especially preferably between 35 and 40 g/g;
j. a moisture of less than 15%, preferably less than 10% and particularly preferably less than 8%;
k. in a 1.0 wt % aqueous solution, a pH value of 3.5 to 5.0 and preferably 3.9 to 4.5;
l. a particle size wherein at least 90% of the particles are smaller than 400 μm, preferably smaller than 350 μm and particularly preferably smaller than 300 μm;
m. a lightness value L*>90, preferably L*>91 and particularly preferably L*>92;
n. a dietary fiber content of the fiber of 80 to 95%.

5. Use according to claim 1, wherein the food product is selected from the group consisting of preserved products, deep-frozen foods, vegan food, vegetarian food, gluten-free food, low-calorie food, low-sugar food, lactose-free food, jelly, jelly-type sweets, sauce, granola bars, fruit pieces, fruit snacks, fruit bars, milk substitute drink, milk substitute product, foam sugar products, sherbet, ice cream, desserts, fermented drink, milk product, delicacies, fruit drink, fruit drink containing alcohol, cocktail, vegetable drink, chutney, barbecue sauce, smoothies, instant drink, fruit spread, fruit compote, fruit dessert, fruit sauce, fruit preparations, bake-stable fruit preparations, fruit preparations for yoghurt, bake-stable vegetable preparations, bake-stable fatty fillings, baked goods, pasta and pasta fillings, noodle dishes, potato snack, cheese and cream cheese preparations, meat substitute products, extruder products, corn flakes, breakfast cereals, soup, sauce, mayonnaise, meats, sausages, sausage casings, seafood, spirits, lozenges, functional food, nutritional supplements and dietary foods such as enteral foods, dysphagia food or sip feed.

6. Use according to claim 1, wherein the feedstuff is selected from the group consisting of feedstuff rich in starches, oleaginous feedstuff, feedstuff rich in protein, extrudate feedstuff, wet feed, binder, bird feedstuff rod, rodent feedstuff rod, fish bait, supplement feedstuff, feedstuff for particular nutritional purposes and dietary feedstuff.

7. Use according to claim 1, wherein the animal need is animal bedding.

8. Use according to claim 1, wherein the sanitary article is selected from the group consisting of diapers, incontinence articles like protective trousers or incontinence pants, sanitary towels, tampons, panty liners and softcups.

9. Use according to claim 1, wherein the personal care product is selected from the group consisting of soap, shower gel, bath additives, skin creams, lotions, gel, sunscreen, sun cream, repellent, shaving cream, shaving soap, epilator cream, toothpaste, dentition adhesive medium, shampoo, hair forming agents, hair-setting products, hair colorants, facial make-up, eye care products, lip care products, nail polish and self-tanning agents.

10. Use according to claim 1, wherein the cleaning agent is selected from the group consisting of detergent, bile soap, washing-up liquid, dishwasher detergent, rinsing agent, neutral cleaner, abrasive cleaner, window cleaner, lime remover, drain cleaner, brake cleaner, alcohol cleaner, all-purpose cleaner, glass cleaner, sanitary cleaner, toilet cleaner, toilet gel, toilet soap, carpet cleaner, car care material, oven cleaner, bathroom cleaner and metal polish, oil absorbing and anti-dust agents.

11. Use according to claim 1, wherein the coating agent is selected from the group consisting of antistatic coating, oil-repellent coating and anti-block coating.

12. Use according to claim 1, wherein the explosive is a gelatinous explosive.

13. Use according to claim 1, wherein the lubricant is selected from the group consisting of liquid lubricant, such as lubricating oil and cooling lubricant, lubricating grease and solid lubricant. Page 7

14. Use according to claim 1, wherein the plastic product is a carrot fiber-reinforced plastic or a wood-plastic composite (WPC).

15. Use according to claim 1, wherein the varnish is selected from the group consisting of alkyd resin varnish, oil varnish, cellulose nitrate varnish, bitumen varnish, tar-containing varnish, phenolic resin varnish, urea resin varnish, melamine resin varnish, polyester varnish, epoxy resin varnish, polyurethane resin varnish, acrylic varnish and powder varnish.

16. Use according to claim 1, wherein the paint is selected from the group consisting of glaze, oil paint, dispersion paint, chalk paint, silicate paint and liquid plaster.

17. Use according to claim 1, wherein the construction material is selected from the group consisting of building foam, sound proofing material, insulation material, concrete, screed, mortar, cement, chemical bonded anchors, chemical anchor bolts, asphalt and whisper asphalt.

18. Use according to claim 1, wherein the adhesive is selected from the group consisting of dispersion adhesive, melt adhesive, plastisol, cyanoacrylate adhesive, methyl methacrylate adhesive, unsaturated polyester adhesive, epoxy adhesive, polyurethane adhesive, silicones, phenolic resin adhesive, polyimide adhesive, polysulfide adhesive, bismaleimide adhesive, adhesive based on silane-modified polymers, silicone adhesive, paste.

19. Use according to claim 1, wherein the fertilizer is a binder for fertilizer cones.

20. Use according to claim 1, wherein the drug is selected from the group consisting of powder, juice, lotion, ointment, cream, gel, tablet and rubber article.

21. Use according to claim 1, wherein the medical product is selected from the group consisting of wound dressing, emergency bandage, transdermal patch, stoma product and dental casting compound.

22. Use according to claim 1, wherein the battery is selected from the group consisting of primary cell, accumulator and solid cell battery.

23. Use according to claim 2, wherein use in the construction area comprises usage in road and path construction, masonry construction, concrete construction and reinforced concrete construction.

24. Use according to claim 2, wherein the use in extraction by drilling boreholes comprises the usage as an additive to a drilling fluid or fracfluid.

25. Use according to claim 2, wherein the use in agriculture comprises the usage in fertilizers, humectants, soil conditioners, plant substrates, flowerpots and substrate-tablet extrudates.

26. Product selected from the group consisting of food products, feedstuff, commodity goods, animal need, sanitary articles, personal care products, cleaning agents, coating agents, care agents, explosives, lubricants, cooling agents, plastic products, fabrics, imitation leather, varnish, ink, paints, building materials, composite materials, paper, cardboard, adhesives, fertilizers, drugs, medical products, batteries, wherein it comprises the activated carrot fiber according to claim 1.

27. Product according to claim 26, wherein the portion of activated carrot fiber is between 0.05 wt % and 90 wt %, preferably between 0.1 and 50 wt %, particularly preferably between 0.1 and 25 wt % and especially preferably between 0.5 and 10 wt %.
